(19) United States
(12) Patent Application Publication
(10) Pub. No.: US 2004/0229347 A1
(43) Pub. Date: Nov. 18, 2004

(54) Embossed test strip system

(76) Inventor: Edward P. Perez, Redwood City, CA (US)

Correspondence Address: WOODARD, EMHARDT, MORIARTY, MCNETT & HENRY LLP, BANK ONE TOWER/CENTER, 111 MONUMENT CIRCLE, SUITE 3700, INDIANAPOLIS, IN, 46204-5137, US

(21) Appl. No.: 10/802,157

(22) Filed: Mar. 17, 2004

Related U.S. Application Data
(63) Continuation of application No. PCT/US02/29327, filed on Sep. 17, 2002.
(60) Provisional application No. 60/323,426, filed on Sep. 17, 2001.

(51) Int. Cl. C12M 1/34; G01N 33/558

(52) U.S. Cl. 435/287.2; 436/514

(57) ABSTRACT

A bodily fluid sampling device includes a support member that defines an aperture to receive a bodily fluid sample and a cover member. A test strip is compressed between the support member and the cover member to form an embossed pillow within the aperture. The embossed pillow is adapted to absorb the bodily fluid sample. The test strip has an incision surrounding the embossed pillow to minimize leakage of the bodily fluid sample from the embossed pillow. The support member can further include a blade that surrounds the aperture to form the incision around the embossed pillow.

### REFERENCE TO RELATED APPLICATIONS

This application is a continuation of International Application No. PCT/US02/29327 (Attorney Docket No. RDID-01099), filed Sep. 17, 2002, published in English, which claims the benefit of U.S. Provisional Application No. 60/323,426 (Attorney Docket No. 01876-397), filed Sep. 17, 2001, which are incorporated by reference in their entirety.

### FIELD OF THE INVENTION

The present invention relates to a system and method for determining the presence or concentration of analytes or biological agents in a sample of bodily fluid using a specific amount of membrane imbibed with dry reagent. In the most preferred embodiment the meter and a single use, reagent bearing test strip is used to measure the concentration of glucose in a bodily fluid such as whole blood or interstitial fluid (ISF).

### BACKGROUND OF THE INVENTION

The need for simple methods to determine the chemical and biological constituents in bodily fluids has increased as point of care testing has gained in popularity. The most common application is the self monitoring of blood glucose concentrations by patients with diabetes. Diabetic patients frequently administer insulin or take other therapeutic actions based on the test results. As testing is generally recommended multiple times daily and may occur in any setting, an easy to use, low sample volume test is required. The issues associated with sample volume are significant to many diabetic patients, especially elderly patients with compromised circulatory systems.

In addition to chronic disease monitoring, there are other applications where simple, low sample size testing at the point of care may be desired. For example, many practitioners believe that certain medications could be administered much more effectively, both from a medical outcome and from a cost perspective, if the circulating level of such medications could be monitored during the course of treatment. Generally, if the level of an analyte or biological agent is important enough, the patient needs to go to a clinic or laboratory and submit to a venipuncture so a test may be run on an expensive clinical instrument. The ability to monitor the patient either in the doctor's office or at home could lead to improved outcomes. By providing a simple low sample volume test, the practitioner is given a means of performing a test utilizing a small sample which in most cases is easier to obtain from the patient by using a simple finger stick.

The National Institute of Health conducted a large scale study to evaluate the benefit of long term tight control of the blood glucose for the diabetic patient. The study, known as the DCCT, proved that long term tight control of the blood glucose levels in patients had a direct relationship to the health of the patient. One way for the medical profession to monitor the control of a patient is for the patient to use a blood glucose monitoring system. One of the main obstacles to testing is the sample size needed to perform the test. As patients age and their circulation decreases, the ability to extract an adequate sample of body fluid is affected. A test which more efficiently utilizes the bodily fluid would aid in reducing the problems associated with larger sample size tests. Current blood glucose monitoring devices such as the One Touch systems manufactured by LifeScan, Inc. of Milpitas, Calif. require the patient to place between 8 and 12 microliters of blood on the test strip. Many patients apply substantially more blood to the test to minimize the failure of the test due to not having enough sample applied to the strip. This unmeasured sample leads to accuracy problems due to more sample than dried chemistry present on the test strip. A system which self meters the amount of sample to a specific amount of carrier consisting of a matrix which holds a relatively constant amount of chemistry and provides a consistent volume for absorbing the sample to promote the test reaction would be a significant advancement to the patient community.

Many diabetics currently use a test method described in U.S. Pat. No. 5,304,468 Phillips et al. This system is comprised of an electronic meter and a disposable reagent strip. The meter reads the color change of the strip which correlates to the concentration of the analyte in the sample applied to the strip. The meter is an expensive and complex instrument which uses multiple light sources or detectors to isolate the reagent color change from the sample color. The user must select the calibration code for the meter to match the calibration code of the test strips. In this way, the meter accommodates a wide range of test strip performance values.

U.S. Pat. No. 4,637,403, to Garcia et al., describes an integrated system which provides a method by which the patient lances the finger to get a sample of blood which is then used by the device to read the quantity of analyte in the sample. This system uses a complex reflectance system to read the analyte level in the sample.

U.S. Pat. No. 5,279,294, to Anderson et al., describes a hand held, shirt pocket device for quantitative measurement of glucose or analytes in biological fluids. The device has a sophisticated electronics system and a sampling system integrated into one device to determine the quantity of analyte in a bodily fluid sample

U.S. Pat. No. 5,515,170, to Matzinger et al., describes the difficulties of keeping a strip holder and optics system clean and the need to present the test strip in the proper perspective to the optics.

European Patent Specification 0 351 891 B1, to Hill et al., describes an electrochemical system and electrodes which are suitable for the in vitro determination of blood glucose levels. The system requires the use of expensive electrodes and a sophisticated reader to determine blood glucose levels.

U.S. Pat. No. 4,994,167, to Shults et al., describes a measuring device for determining the presence and amount of a substance in a biological fluid using electrochemical methods. This system requires a complex instrument and method for the patient to determine the quantitative result.

U.S. Pat. No. 5,580,794, to Allen et al., describes a single use disposable measuring device for determining the presence and amount of a substance in a biological fluid using reflectance methods. This system utilizes optics and electronics packages which are mated in a single plane.

Single use disposable devices have been designed for the analysis of analytes in bodily fluids. U.S. Pat. No. 3,298,789, to Mast, describes a system in which whole blood is applied to a reagent strip. After a precise, user-timed interval, the blood must be wiped off by the user. An enzyme system reacts with the glucose present in the sample to create a color change which is proportional to the amount of glucose in the sample. The strip may be read visually by comparing to a printed color intensity scale, or in an electronic instrument.

U.S. Pat. No. 5,418,142, to Kiser et al., describes a single use device which does not require blood removal or color matching. The amount of analyte present in the sample is read in a semiquantitative fashion.

U.S. Pat. No. 5,962,215, to Douglas et al., describes a series of semiquantitative, single use devices which are used to determine the level of an analyte in a biological sample. These devices do not require blood removal or color matching.

U.S. Pat. No. 5,451,350, to Macho et al., describes a single use system for the determination of an analyte in a biological sample.

European Patent Application No. EP 0 759 555 A2, to Douglas et al., describes a multilayer reagent test strip which measures the concentration of analyte in a liquid sample that is applied to it.

U.S. Pat. No. 4,994,238, to Daffern et al., describes a multilayer test device which uses a defined area of absorbent, reagent bearing matrix.

Although many improvements have been made, the cost and complexity of measuring analyte levels in biological samples remains a significant issue for patients and for the health care system. The need to deliver a sizable sample of bodily fluid to the strips or electrodes in use leads to errors in performance and presents problems for the patient. The availability of a low sample volume which meters the sample to the test matrix reduces the issues with short sampling or over sampling of the test. This is a great advantage to the patient to insure an accurate test. A simplified quantitative test system of this invention for the periodic monitoring of constituents of biological fluids, such as glucose in blood, would make testing more accessible to patients and would improve their well-being.

A system which requires a smaller fluid sample is attractive to many patients. There has been a trend toward smaller sample sizes, but most devices still require about 10 μL of blood. Many patients have difficulty routinely applying an adequate sample to the strips or electrodes. Inadequate sampling can cause erroneous results or may require that the user discard an expensive test strip and repeat the sample application procedure. A system which would require about 3 μL or less, which is a fraction of the volume required for most blood glucose tests and could be more readily obtained by patients, would be advantageous.

An object of the present invention is to provide a method for measuring the amount of analyte in a sample of biological fluid using a simple, low sample volume, reagent test strip with a built in metering system.

Another object of this invention is to provide reagent test strips that can meter the sample into the reaction matrix.

### SUMMARY OF THE INVENTION

The method of this invention involves the use of single use test strips capable of reading small sample sizes and determining the amount of an analyte in the small sample. The low sample size feature of the strip permits the patient to use less invasive systems to acquire a sample than the 21 gauge lancing devices in current use. The device is structured with a capillary to meter a specific quantity of sample to the test matrix, thereby eliminating a significant source of error associated with short sampling. The capillary is designed so that, when placed in contact with a sample of bodily fluid, it transfers the sample to the test matrix. If the sample is insufficient to travel the full length of the capillary, then the sample does not reach the test matrix and will not wick into the test matrix, which prevents the patient from short sampling the test strip. The user can add additional sample to the capillary to complete the test. Once the sample contacts the test matrix, the sample will wick into the test matrix until the test matrix is filled, then stop. Excess sample remains in the capillary and serves as a signal to the patient that the test matrix has the correct amount of sample for the test. This provides many advantages to the patient including the elimination of wasted strips due to short sampling which results in a substantial cost savings for the patient and reduces the number of inaccurate tests from marginal samples.

The capillary design also provides another interesting benefit. As blood travels down through the capillary to the test area, the blood warms the peg, thus regulating the temperature of the strip and the test. This is beneficial in two ways; the first is that each test is performed under somewhat controlled conditions, regardless of whether or not the surrounding temperature is warm or cold. Second, this effect alleviates the problem of fogging over the test area. This is a problem with many blood glucose monitors when testing in cooler ambient conditions.

The formation of a captivated microtitration zone is described in U.S. Pat. No. 5,872,713 Douglas et al. When constructed according to this invention, the microtitration zone can be achieved with a specific volume by following a simple series of steps: (a) applying a specific amount of reagent such that it does not saturate the matrix and is developed to indicate a specific analyte, (b) drying the reagent so that the active ingredients adhere to the substrate of the matrix, (c) embossing or compressing the matrix to collapse the matrix surrounding the reaction zone so that the void volume of the resulting test matrix microtitration volume is approximately equal to the sample size desired, (d) installing it into a performed pocket which completely surrounds all the circumference of the pillow where the capillary is in communication with the top side/sample side of the pillow, and (e) sealing the system together. The embossed/collapsed areas have had their void volume reduced to approximately zero and the test matrix reaction zone forms a small bibulous pillow which retains its void volume and has the desired total volume. This limits the ability of the reagents imbibed into the embossed matrix to participate in the reaction of the result zone. The test pad can be made from various matrix materials which will hold the test reagent in a dried form including polyethersulphone (Gelman sciences Supor 200D), polysulphone (Memtec filtration asymmetric membrane) and nylon (Pall biodyne). The wicking material which can be selected from various materials, including Pall Accuwick and Whatman 41, which provide a high enough capillary action to wick and absorb the sample from the capillary peg and spread it into and fill the reaction matrix microtitration volume.

The applied bodily fluid reacts with the reagents impregnated in the test pad within the test strip and the resulting color change is read by the optics system of the meter adapted to read the strip.

The patient uses the test strip by removing it from the packaging and placing it into a meter designed to utilize the test strip. The patient turns the meter on or it can be automatically started from the test strip insertion. The patient uses either a sampler from the kit or one procured separately to draw a sample of capillary blood. This sample is applied to the test strip, the meter reads the sample, and the meter displays the result after an appropriate time.

One aspect of the present invention concerns a bodily fluid sampling assembly. The assembly includes a support member that defines an aperture adapted to receive a bodily fluid sample and a cover member. A test strip is compressed between the support member and the cover member to form an embossed pillow within the aperture. The embossed pillow is adapted to absorb the bodily fluid sample. The test strip has an incision surrounding the embossed pillow to minimize leakage of the bodily fluid sample from the embossed pillow.

Another aspect of the present invention concerns a test strip assembly that includes a wicking layer for collecting a bodily fluid sample and a support member. The support member defines an opening and has a blade extending around the opening. The blade contacts the wicking layer to minimize flow of the fluid sample in the wicking layer from the opening.

A further aspect of the present invention concerns a test strip. The test strip includes a test matrix to test a bodily fluid sample. A wicking layer is provided over the test matrix. The wicking layer has an embossed pillow for absorbing the bodily fluid sample. The wicking layer has an incision surrounding the embossed pillow to minimize leakage of the bodily fluid sample from the embossed pillow.

Other forms, embodiments, objects, features, advantages, benefits and aspects of the present invention shall become apparent from the detailed drawings and description contained herein.

### BRIEF DESCRIPTION OF DRAWINGS

FIG. 1 is an exploded, side elevation view of one embodiment of a test pad matrix and wicking layer prior to being embossed in a die formed by plates.

FIG. 2 is a cross-sectional view of one embodiment of a test pad matrix and wicking layer during embossing in a die formed by plates.

FIG. 3 is an exploded perspective, cut away view of the test pad matrix, wicking layer and upper and lower plates of the embossing die.

FIG. 4A is an assembled view and 4B is an exploded perspective view of one embodiment of the strip showing assembly of the handle, test pad, wicking layer, and capillary.

FIG. 5 is an enlarged, cross-sectional view of a test strip constructed according to the present invention.

FIG. 6 is an exploded, cross-sectional view of an alternative embodiment of the test strip in accordance with the present invention.

FIG. 7 is a cross-sectional side view of the alternative embodiment of the test strip as assembled.

FIG. 8 is an exploded, cross-sectional side view of another alternative embodiment of a test strip in accordance with the present invention.

FIG. 9 is a cross-sectional side view of the test strip of FIG. 8 as assembled for use.

FIG. 10 is an enlarged, cross-sectional view of a test strip constructed according to another embodiment of the present invention.

### DESCRIPTION OF SELECTED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. One embodiment of the invention is shown in great detail, although it will be apparent to those skilled in the art that some of the features which are not relevant to the invention may not be shown for the sake of clarity.

The present invention provides improvements over existing technology in use today in several ways. A preferred embodiment of the invention creates a microtitration zone which permits the accurate testing of a small fluid sample and prevents oversampling, while the integrated capillary provides a means to eliminate the problems associated with short sampling which frequently occurs in the current commercial products. The capillary also provides a means of absorbing the fluid sample from a non-fingerstick location. This permits the use of non-traditional lancing systems. The small test pad used in this invention reduces the cost of the matrix employed and the quantity of expensive reagents needed to conduct an accurate assay using an oxidase and peroxidase chemistry. With a smaller test pad, a smaller sample volume is adequate. It should be noted also that an electrode based test system could be used with the basic structure and elements of this invention. A further feature of the capillary is that the capillary acts as a retaining chamber where a sample of appropriate volume is initially collected and then delivered to the test pad. The sample is only delivered to the test pad when a sufficient amount of sample has been collected within the capillary. Furthermore, the capillary may be constructed to further include a fluid chamber. The fluid chamber may be disposed adjacent the test media, such that in use, the distal end of the capillary is placed in contact with a bodily fluid to be sampled. The bodily fluid is received within the capillary through capillary action or other means such as a wicking material. As bodily fluid is drawn into the capillary the bodily fluid fills the fluid chamber. After the fluid chamber is filled, the sample of bodily fluid collected in the fluid chamber may then be deposited upon the test media. A benefit of using a fluid chamber is that a smaller sized sample may be utilized to perform a desired test because the entire amount of bodily fluid needed for the test can be accurately delivered to a test site, thereby reducing the overall amount of sample needed to perform the test. Furthermore, the use of a fluid chamber to collect the sample may also lead to fewer failed tests due to inadequate sample volume, because the sample will not be delivered to the test media until a sufficiently sized sample is collected.

Although the fluid chamber has been described in use with a capillary, it is contemplated that other collection devices may be utilized with the fluid chamber of the present invention. For example, the fluid chamber may be included within a test strip wherein the sample is placed on a portion of the test strip and transported to the fluid chamber. The descriptions above should not be considered limiting and are intended to be exemplary.

The test strip is comprised of a test pad situated in a test pad holder. This holder provides a means for accurately positioning the test pad with respect to the optics system in the meter and for providing a means for blocking ambient light from affecting the analysis. The test pad is impregnated with the appropriate chemistry to permit a calorimetric analysis of the analyte being tested, and must therefore provide a stable absorbent substrate. If the system is developed with an electrode based system, the function of the test pad holder is to position the electrode contacts on the strip with those corresponding to the meter. The test pad can be made from various materials which will hold the test reagent in a dried form, including polyethersulphone (Gelman Sciences Supor 200D), polysulphone (Memtec filtration asymmetric membrane) and nylon (Pall Biodyne). The wicking layer can likewise be selected from various materials, including Pall Accuwick and Whatman 41, which provide a high enough capillary action to absorb the sample and spread it to the reaction matrix.

The test strip of this invention provides a support for the test pad and the capillary peg contacting the test pad. The peg positively seats in the meter in a detent and is locked from rotation by a corresponding key in the test strip which fits into a slot in the meter test strip holder. The test strip holder is positioned to the optics block using pins on the optics block assuring proper alignment of the test strip. It also seals the optics area from ambient light and any excess blood contamination. These features are more fully disclosed in U.S. Pat. No. 5,872,713, which is incorporated herein by reference.

The signal producing system impregnated in the test pad matrix can be formed from different indicator systems, such as 3-methyl-2-benzothiazolinone hydrazone (MBTH) and 8-anilino-1-naphthalenessulfonate(ANS) [U.S. Pat. No. 5,453,360 to Yu], MBTH and 3-dimethylaminobenzoic acid (DMAB) [U.S. Pat. No. 5,049,487 to Phillips et al.], 3-methyl-2-benzothiazolinone-hydrazone-sulfonate sodium salt (MBTHS) and -Ethyl-N-(3-sulfopropyl)aniline (ALPS) [U.S. Pat. No. 4,396,714 to Maeda et al.]. One skilled in the art could devise an alternate indicator system. The oxidase enzyme system contained in the reagent pad produces hydrogen peroxide which is used to convert the indicator with the assistance of peroxidase which acts as the catalyst.

In the most preferred embodiment the reagents are impregnated into a porous membrane by submerging the dry membrane into a reagent dip. Excess fluid is wiped from the membrane surface and the membrane is gently dried in an oven. At this point, subsequent dipping and drying can be conducted. A preferred embodiment for a two dip process is:

MBTHS & ALPS Formulation

Final
Concentrations

A Dip

| | |
|---|---|
| In Citrate buffer, pH 7 | 0.1 M |
| stock A Dip | |
| EDTA | 0.08% |
| mannitol | 0.19 |
| Gantrez-S95 | 0.53% |
| Klucel 99-EF | 20 uM |
| Crotein-SPA | 7.45% |
| enzyme reagents | |
| Glucose Oxidase | 0.92% |
| Peroxidase | 0.54% |

B Dip

| | |
|---|---|
| In 70% Ethanol | |
| MBTHS | 0.66% |
| ALPS | 2.00% |
| SOS | 0.20% |

The color formed after applying the bodily fluid to the reagent test pad is proportional to the amount of analyte in the applied sample. The meter measures the change in reflectance due to the development of the specific color generated by the indicator. This is either used as the input to a function which relates reflectance to analyte level or to a table which correlates reflectance value to analyte level. The function or the table is stored within the meter system for it to produce and display a reading of the analyte level. While most meters in use today employ functions to convert reflectance readings to analyte concentration, this approach requires that the function be stable and well understood. The use of a look up table permits the storage of specific values for reflectance and their corresponding analyte levels. The meter uses this table and interpolates between the table values to give relatively accurate readings. This is achievable in a system such as that described by this invention as the table can quickly be generated for each reagent lot produced. The devices of this invention using a read-once calibration chip, or being fully disposable, can use a lot-specific look up table to convert reflectance reading to analyte levels.

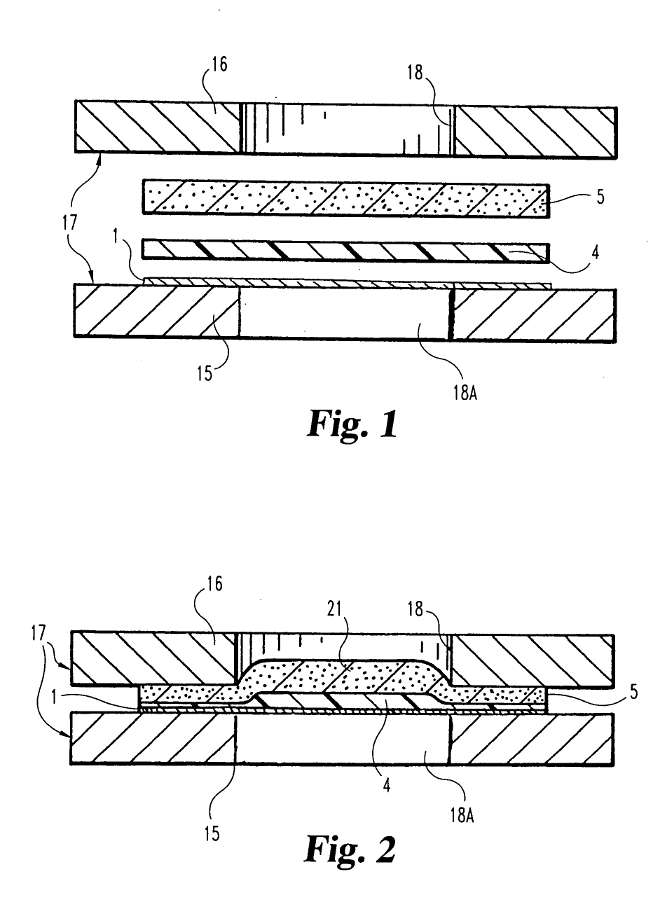

FIG. 1 shows an elevation view of the un-embossed layers, wicking layer 5, test matrix layer 4, and top layer 1 between the die 17 formed from top plate 16 containing hole 18 and bottom plate 15 containing hole 18A.

FIG. 2 shows an elevation view of the embossed or compressed layers, wicking layer 5, test matrix layer 4, and top layer 1 between the die 17 formed from top plate 16 containing hole 18 and bottom plate 15 containing hole 18A. Hole 18 in die plate 16 forms the microtitration pillow 21 in the wicking layer 5 and in test matrix layer 4. The areas of the layers surrounding pillow 21 are compressed to make them essentially impervious to sample liquid flow, thus forming the microtitration volumetric area around pillow 21. Hole 18A allows for the test strip to be placed in an optical meter whereby a color change of the top layer and/or matrix layer can be measured.

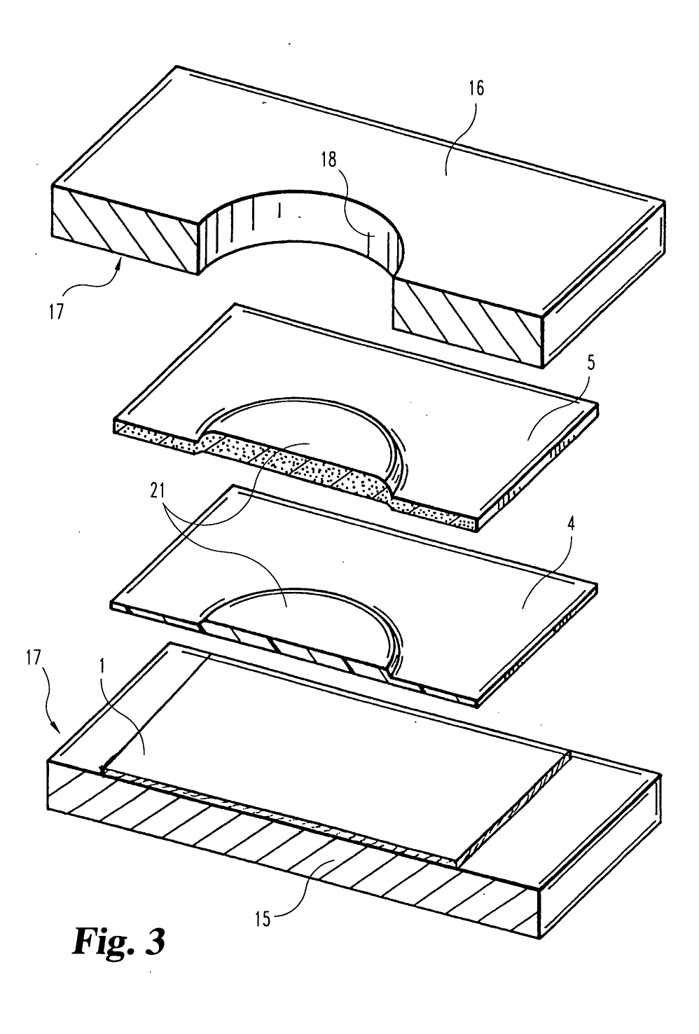

FIG. 3 shows an exploded perspective view of the embossed or compressed layers, wicking 5, test matrix 4, and top layer 1 as formed between the die 17 formed from top plate 16 and bottom plate 15 containing hole 18A.

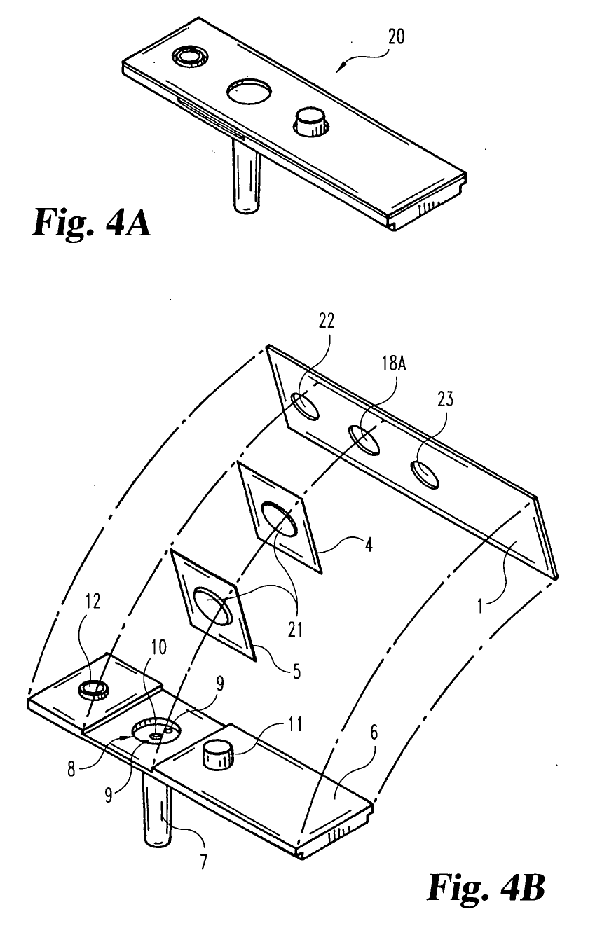

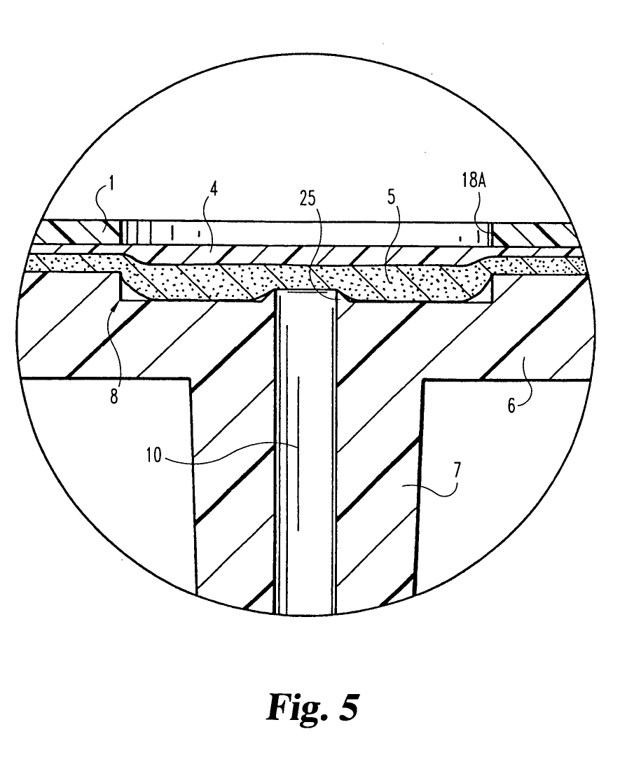

The assembly of a test strip 20 shown in FIG. 4A is accomplished as shown in FIG. 4B. In a preferred embodiment bottom or support member 6 which has the capillary peg 7 and capillary 10 integrally molded therein (e.g., by injection molding) and constructed so that microtitration pocket 8 has breather holes 9 located within the microtitration pocket 8. Or capillary peg 7 can be formed as a separate element and assembled into support member 6 if desired. FIG. 2 shows the formation of the microtitration pillow 21 in matrix 4 and wicking layer 5. The microtitration pillow 21 is formed using die 17 formed from top plate 16 and bottom plate 15. By using a die to form the pillows the spacing of the pillows 21 can be formed in the matrix 4 and wicking 5 so that they align with the microtitration pocket 8. When the top layer 1 is assembled on bottom member 6 with test matrix layer 4 and wicking layer 5 properly positioned as shown between layers 1 and 6. Test matrix pad 4 is formed from a bibulous matrix which has been impregnated with a reagent system comprised of enzymes, indicators and blood separation agents and the wicking matrix pad 5 provides a means of spreading the sample over the test pad 4. Layers or pads 4 and 5 are preferably embossed or compressed prior to assembly with layers 1 and 6. The holes 22 and 23 formed in the top layer 1 and alignment keys 11 and 12 formed in holder 6 provide a means of aligning the test strip assembly including pillow 21 and hole 18A to the microtitration pocket 8. The breather holes 9 provide an escape path for trapped air in the assembly pillow 21 when wicking the sample up the capillary 10 and into pillow 21. FIG. 5 shows an additional preferred feature of the present invention where capillary peg 7 and capillary tube 10 are formed with a protruding collar 25 extending from capillary tube 10 to engage and further compress pillow 21. This feature provides a seal between capillary tube 10 and the surface of wicking layer 5, which better forces the sample flow from capillary tube 10 into the interior of wicking layer 5 to better distribute the sample throughout test matrix layer 4 and completely fill microtitration volume 8 and to better prevent sample from flowing between the surface of wicking layer 5 and the surface of the end of capillary peg 7.

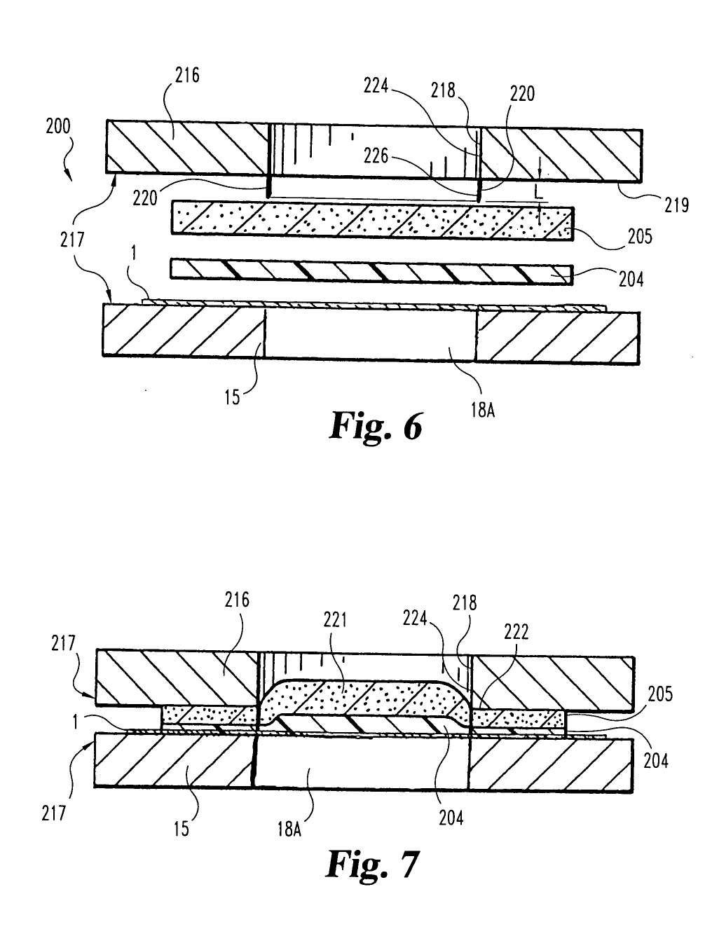

FIGS. 6 and 7 illustrate a test strip 200 according to another embodiment of the present invention. The test strip 200 includes a top layer 1, a test matrix 204 and a wicking layer 205 of the type as described above. As shown, the test matrix 204 is sandwiched between the top layer 201 and the wicking layer 205. Layers 204 and 205 as well as the test matrix 204 are pressed between a bottom plate (cover member) 15, and a top plate (support member) 216. When pressed together, plates 15 and 216 form a die 217 with plates 15 and 216 each defining openings 18A and 218, respectively. In one embodiment, openings 18A and 218 have a generally cylindrical shape. However, as should be appreciated, openings 18A and 218 can be shaped differently. Bodily fluid samples are collected through the opening 218 in the top plate 216, and the opening 18A in the bottom plate 15 allows the sample collected on the test matrix 204 to be analyzed.

As shown in FIG. 7, plate 216 has an interior surface 219 with a blade member 220 projecting therefrom towards plate 15. In one embodiment, the blade 220 is integrally formed with the top plate 216, and in another embodiment, the blade 220 is a separate component attached to plate 216. During assembly, the top plate 216 and the bottom plate 115 are compressed to make the portion of the wicking layer 205 and the test matrix 204 adjacent the aperture 218 virtually impervious to a bodily fluid sample. Within the opening 218 in plate 216, the wicking layer 205 and the test matrix 204 are embossed to form a microtitration area or pillow 221, which is able to absorb the fluid sample. Moreover, the blade 220 in one embodiment presses into the wicking material 205 and is sufficiently sharp to form an incision or cut 222 at least through part of the wicking layer 205 in order to minimize the amount of fluid leakage from the microtitration pillow 221. In another embodiment, the blade 220 only compresses the test strip 200 around the microtitration pillow 221 making the periphery of the micortitration pillow 221 impervious to fluid so as to minimize fluid leakage from the microtitration pillow 221. The blade 220 in the illustrated embodiment has a generally cylindrical shape. However, it should be appreciated that the blade 220 can be shaped differently.

In one embodiment, the blade 220 has a length L that is sized to only cut the incision 222 through part of the wicking layer 205 so that the microtitration pillow 221 of the wicking layer 205 remains attached to the remainder of the test strip 200. In another embodiment, the blade 220 is sized to cut the incision 222 completely through the wicking layer 205. The microtitration pillow 221 of the wicking layer 205 in one form of this embodiment bonded to the test matrix 204, and in another form, the microtitration pillow 221 of the wicking layer 205 is retained in opening 18 through frictional engagement. In the illustrated embodiment, the blade 220 has a closed, continuous shape so that the incision 222 encircles the microtitration pillow 221. Although the incision 222 in the illustrated embodiment is continuous to minimize fluid leakage from the microtitration pillow 221, it should be understood that the incision 222 can be formed in a discontinuous manner such that fluid leakage prevention is not severely compromised. For example, the blade 220 in another embodiment can include cut out sections that form retaining webs in the wicking layer 205 such that the formed incision 222 is discontinuous.

In the illustrated embodiment, the blade 220 has a generally cylindrical shape to coincide with the shape of opening 218. As depicted in FIG. 6, inner surface 224 of opening 218 is flush with inner surface 226 of the blade. With the blade 220 being flush with the opening 218, the incision 222 is formed at the periphery of the microtitration pillow 221 in order to effectively destroy the wicking function of the material adjacent the incision 222. This in turn minimizes leakage of the fluid sample from the microtitration pillow 221. Minimizing leakage from the microtitration pillow 221 reduces the amount of fluid required for the fluid sample. Therefore, as a sample of bodily fluid is placed upon the microtitration pillow 221, the wicking layer 205 distributes the sample across the area of the opening 218, and the blade 220 acts to prevent any sample from flowing beyond it. Since the blade 220 prevents fluid from passing outside the area of the aperture 218, less fluid needs to be collected.

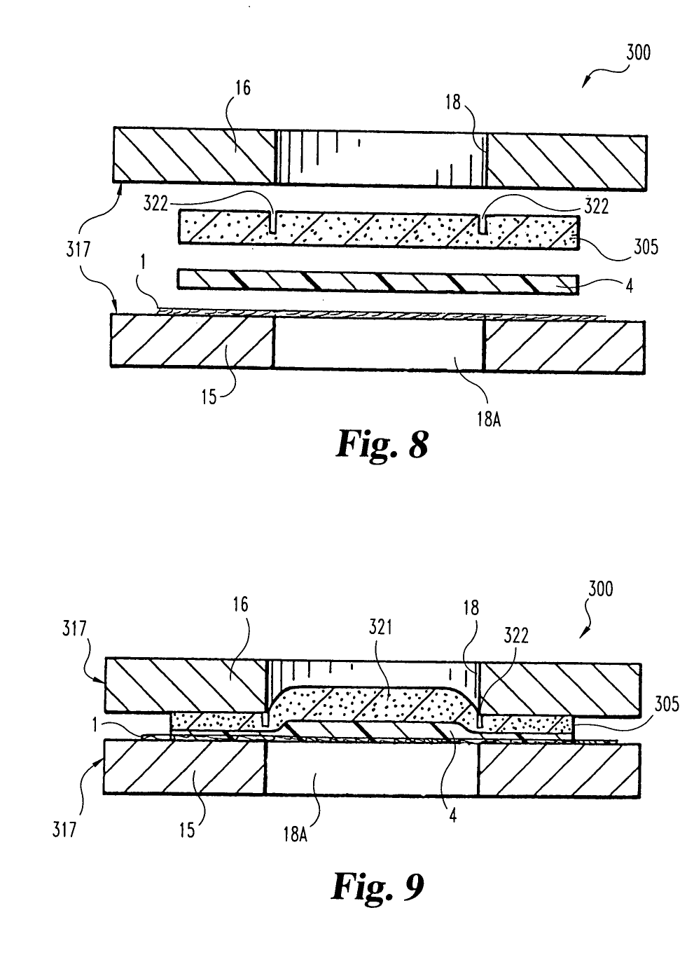

Referring to FIGS. 8 and 9, a test strip 300 according to another embodiment of the present invention includes top layer 1, test matrix layer 4 and a wicking layer 305, which is sandwiched between bottom plate 15 and top plate 16. The top plate 16, as illustrated, defines aperture or opening 18, and the bottom plate 15 defines aperture or opening 18A. In the illustrated embodiment, a fluid sample is collected on the test strip 300 through opening 18. Opening 18A enables a test device to perform a measurement upon the sample collected the test strip 300, such as a colormetric measurement in which the reflectance of the collected sample is measured in order to determine the amount of glucose in the sample.

In one embodiment, the wicking layer 305 and test matrix layer 4 are pre-embossed so as to form a microtitration pillow 321. In another embodiment, the microtitration pillow 321 is formed in the test strip 300 in opening 18 when the test strip 300 is pressed between the top plate 16 and the bottom plate 15 (FIG. 9). In the illustrated embodiment, the wicking layer 305 has an incision 322 formed therein before assembly with the other layers of the test strip 300. As shown, the incision 322 only partially cuts through the wicking layer 305. It should be appreciated that in other embodiments the incision 322 can be formed completely through the wicking layer 305 and/or the test matrix 4. In one form, the incision 322 is pre-cut with a blade before assembly. However, as should be understood, the incision 322 can be fabricated in other manners. As depicted in FIG. 9, the incision 322 is formed to align with and generally correspond with the shape of the opening 18 in the top plate 16 so that the incision 322 surrounds the microtitration pillow 321 formed in the test strip 300.

The incision 322 formed in the wicking layer 305 interrupts fluid flow in the wicking layer 305 to the area surrounding the microtitration pillow 321. In use, a sample of bodily fluid is placed upon the wicking layer 305 whereby the sample spreads across the microtitration pillow 321. The incision 322 prevents the bodily fluid from flowing past the microtitration pillow 321 defined by the aperture 18. By not allowing the fluid sample to flow beyond the aperture 18, less fluid is wasted so that a smaller fluid sample is needed.

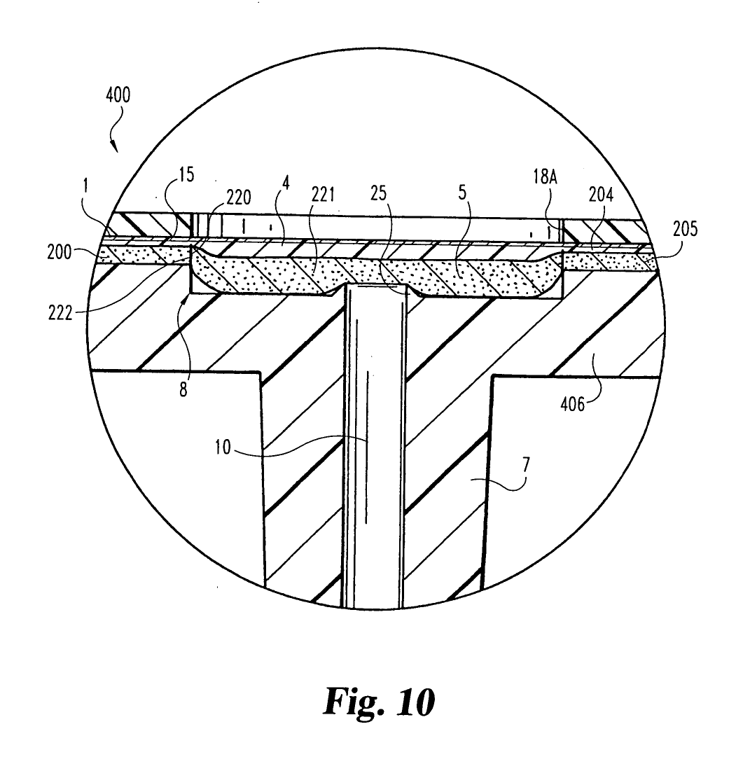

FIG. 10 illustrates an enlarged view of a bodily fluid sampling assembly 400 incorporating test strip 200, according to another embodiment of the present invention. As illustrated, assembly 400 includes a support member 406 that has capillary peg 7, cover member 15, and test strip 200 sandwiched between the support member 406 and the cover member 15. The support member 406 defines a microtitration pocket 8 that fluidly communicates with a capillary tube 10 integrally formed within the capillary peg 7. The capillary tube 10 is used to draw a bodily fluid sample into the microtitration pocket 8. In the illustrated embodiment, the capillary peg 7 is integrally formed with the support member 406. Nonetheless, it should be understood that the capillary peg 7 can be formed as a separate component and attached to the support member 406. In FIG. 10, the support member 406 has a protruding collar 25 that extends from the capillary tube 10 in the microtitration pocket 8 in order to compress the test strip 200. The support member 406 further includes a blade member 220 that surrounds and is aligned with the periphery of the microtitration pocket 8. The cover member 15 defines a sensor aperture 18A that is aligned with the microtitration pocket 8.

As described above, the test strip 200 includes top layer 1, test matrix 204 and wicking layer 205. When the test strip 200 is pressed between the support member 406 and the cover member 15, a microtitration pillow 221 in the test strip 200 is formed within the microtitration pocket 8. The protruding collar 25 engages and compresses the microtitration pillow 221 to improve the seal between the capillary tube 10 and the wicking layer 205. This configuration improves distribution of the bodily fluid sample within the test matrix 204. Further, in the illustrated embodiment, the blade 220 forms an incision 222 in the test strip 200 around the microtitration pillow 221. As discussed above, this incision 222 reduces the amount of bodily fluid required for a sample because less of the fluid is wasted by leaking from the microtitration pillow 221. In another embodiment, the blade 220 does not form the incision 222 in the test strip 200. Instead, the blade 220 compresses the periphery of the microtitration pillow 221 in order to make the periphery of the pillow 221 substantially impervious to fluid so as to minimize the amount of fluid required for a sample.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. A test strip assembly, comprising:
a support member defining an aperture to receive a bodily fluid sample;
a cover member;
a test strip compressed between said support member and said cover member to form an embossed pillow within said aperture, said embossed pillow being adapted to absorb the bodily fluid sample; and
wherein said test strip has an incision surrounding said embossed pillow to minimize leakage of the bodily fluid sample from said embossed pillow.

2. The test strip assembly of claim 1, wherein said support member has a blade member surrounding said aperture to form said incision in said test strip.

3. The test strip assembly of claim 1, wherein:
said support member includes a capillary tube to collect the bodily fluid sample; and
said aperture includes a microtitration pocket defined in said support member.

4. The test strip assembly of claim 1, wherein said test strip includes:
a wicking layer adapted to absorb the bodily fluid sample;
a test matrix for reacting with the bodily fluid sample; and
wherein said incision is formed in said wicking layer.

5. The test strip assembly of claim 4, wherein said incision is further formed through said wicking layer and said test matrix.

6. The test strip assembly of claim 4, wherein said test strip includes a top layer positioned between said top member and said test matrix.

7. A test strip assembly, comprising:
a wicking layer to collect a bodily fluid sample; and
a support member defining an opening, said support member having a blade member extending around said opening and contacting said wicking layer to minimize flow of the fluid sample in said wicking layer from said opening.

8. The test strip assembly of claim 7, wherein:
said wicking layer defines an incision that coincides with said opening to minimize flow of the fluid sample in said wicking layer from said opening; and
said blade member is adapted to cut said incision in said wicking layer.

9. The test strip assembly of claim 7, wherein said blade member compresses said wicking layer to minimize flow of the fluid sample in said wicking layer from said opening.

10. The test strip assembly of claim 7, further comprising a capillary tube provided over said wicking layer to deliver the bodily fluid sample to said wicking layer.

11. The test strip assembly of claim 7, further comprising:
a top layer; and
a test matrix positioned between said top layer and said wicking layer for analyzing the bodily fluid sample.

12. The test strip assembly of claim 7, wherein said wicking layer has a microtitration pillow surrounded by said blade member.

13. A test strip, comprising:
a test matrix to test a bodily fluid sample; and
a wicking layer provided over said test matrix, said wicking layer having an embossed pillow for absorbing the bodily fluid sample, said wicking layer having an incision surrounding said embossed pillow to minimize leakage of the bodily fluid sample from said embossed pillow.

14. The test strip of claim 13, further comprising a top layer covering said test matrix.

15. The test strip of claim 13, wherein said incision has a circular shape.

16. The test strip of claim 13, wherein said incision is continuous.
